(19) United States
(12) Patent Application Publication
(10) Pub. No.: US 2014/0212568 A1
(43) Pub. Date: Jul. 31, 2014

(54) DURUM WHEAT PLANTS HAVING A PARTIALLY OR FULLY MULTIPLIED GENOME AND USES THEREOF

(71) Applicants: Amit Avidov, Kiryat-Tivon (IL); Alon Lerner, Moshav Sharona (IL); Itamar Lupo, Moshav Elad (IL); Limor Baruch, Moshav Tel Adashim (IL)

(72) Inventors: Amit Avidov, Kiryat-Tivon (IL); Alon Lerner, Moshav Sharona (IL); Itamar Lupo, Moshav Elad (IL); Limor Baruch, Moshav Tel Adashim (IL)

(73) Assignee: Kaiimam Bio Agritech Ltd., Kfar-Tavor (IL)

(21) Appl. No.: 14/238,509

(22) PCT Filed: Aug. 13, 2012
(86) PCT No.: PCT/IL2012/050309

§ 371 Date: Feb. 12, 2014

Related U.S. Application Data
(60) Provisional application No. 61/523,353, filed on Aug. 14, 2011.

(51) Int. Cl. A01H 5/10; A23L 1/10; A23K 1/14

(52) U.S. Cl. CPC A01H 5/10; A23K 1/14; A23L 1/1041
USPC 426/622; 800/320.3; 435/410; 800/260; 800/276; 426/627; 426/623

(57) ABSTRACT

A Durum wheat plant having a partially or fully multiplied genome being at least as fertile as a tetraploid Durum wheat (Triticum Durum) plant isogenic to said genomically multiplied Durum wheat plant when grown under the same conditions and being of the same developmental stage is provided. Also provided are methods of generating and using same as well as products generated therefrom.

### FIELD AND BACKGROUND OF THE INVENTION

The present invention, in some embodiments thereof, relates to Durum wheat plants having a partially or fully multiplied genome and uses thereof.

Durum wheat or macaroni wheat (also spelled as Durhum; or known as Triticum durum or Triticum turgidum durum) is the only tetraploid species of wheat of commercial importance that is widely cultivated today. Durum wheat has 28 chromosomes originating through intergeneric hybridization and polyploidization involving two diploid grass species: T. urartu (2n=2x =14, AA genome) and a B-genome diploid related to Aegilops speltoides (2n=2x=14, SS genome) and is thus an allotetraploid species

Among all cultivated wheats, Durum wheat and Bread wheat are the most important cereal crops in the world. Durum wheat is a minor crop, grown on only 8 to 10% of all the wheat cultivated area. The remaining area is cultivated with hexaploid bread wheat.

Durum wheat is better adapted to semiarid climates than is bread wheat. The world's durum wheat acreage and production is concentrated in the Middle East, North Africa, the former USSR, the North American Great Plains, India, and Mediterranean Europe. Durum is a spring wheat, although winter durum is grown. In spite of its low acreage, durum wheat is an economically important crop because of its unique characteristics and end products. It is generally considered the hardiest of all wheats. Durum kernels are usually large, golden amber, and translucent.

These characteristics, along with its protein content and gluten strength, make it suitable for manufacturing diverse food products. Pasta is the most common durum end product consumed in Europe, North America, and the former USSR. Products other than pasta are also made from durum wheat. Couscous, made from durum semolina, is consumed mainly in North Africa. Flat bread made from durum wheat and bulgur is part of the main diet in Jordan, Lebanon, and Syria.

The quality of Durum wheat is highly correlated with the quality of its end products. Durum wheat, with its high kernel weight, test weight, protein content, and gluten strength, is known to be associated with the firmness and resiliency of the cooked pasta products and the stability of cooking.

Due to the commercial importance of Durum wheat, various Durum plant breeding and genetics programs were developed. Cultivars released from North Dakota's breeding program are grown on over 93% of Durum hectares in North Dakota and surrounding states.

Additional Background Art Includes:

U.S. Pat. Application Number 20030005479 teaching methods of chromosome doubling.

Perak An. Inst. Fitotec. 1940 Vol. 2 pp. 7 reported the injection of colchicine into the coleoptile. Plants with enlarged stomata were obtained in T. durum, T. pyramidale and T. Timopheevi. Only that from T. durum reached maturity; it was highly sterile and produced only four seeds. From these, two plants were obtained, both having 56 chromosomes in the root tips. No polyploids were obtained by treating the seed or the spike.

### SUMMARY OF THE INVENTION

According to an aspect of some embodiments of the present invention there is provided a Durum wheat plant having a partially or fully multiplied genome being at least as fertile as a tetraploid Durum wheat (Triticum durum) plant isogenic to the genomically multiplied Durum wheat plant when grown under the same conditions and being of the same developmental stage.

According to an aspect of some embodiments of the present invention there is provided a hybrid plant having as a parental ancestor the partially or fully genomically multiplied plant.

According to an aspect of some embodiments of the present invention there is provided a hybrid Durum wheat plant having a partially or fully multiplied genome.

According to an aspect of some embodiments of the present invention there is provided a planted field comprising the partially or fully genomically multiplied plant.

According to an aspect of some embodiments of the present invention there is provided a sown field comprising seeds of the partially or fully genomically multiplied plant.

According to some embodiments of the invention, the partially or fully genomically multiplied plant is non-transgenic.

According to some embodiments of the invention, the partially or fully genomically multiplied plant has a spike number at least as similar to that of the tetraploid Durum wheat (Triticum durum) plant under the same developmental stage and growth conditions.

According to some embodiments of the invention, the partially or fully genomically multiplied plant has a spike width at least as similar to that of the tetraploid Durum wheat (Triticum durum) plant under the same developmental stage and growth conditions.

According to some embodiments of the invention, the partially or fully genomically multiplied plant has a spikelet number at least as similar to that of the tetraploid Durum wheat (Triticum durum) plant under the same developmental stage and growth conditions.

According to some embodiments of the invention, the partially or fully genomically multiplied plant has a spike length at least as similar to that of the tetraploid Durum wheat (Triticum durum) plant under the same developmental stage and growth conditions.

According to some embodiments of the invention, the partially or fully genomically multiplied plant has grain weight at least as similar to that of the tetraploid Durum wheat (Triticum durum) plant under the same developmental stage and growth conditions.

According to some embodiments of the invention, the partially or fully genomically multiplied plant has grain yield per plant at least as similar to that of the tetraploid Durum wheat (Triticum durum) plant under the same developmental stage and growth conditions.

According to some embodiments of the invention, the partially or fully genomically multiplied plant has grain yield per area at least as similar to that of the hexaploid common wheat (Triticum durum) plant under the same developmental stage and growth conditions.

According to some embodiments of the invention, the partially or fully genomically multiplied plant has grain size similar to that of the tetraploid Durum wheat (Triticum durum) plant under the same developmental stage and growth conditions.

According to some embodiments of the invention, the partially or fully genomically multiplied plant has grain protein content similar to that of the tetraploid Durum wheat (Triticum durum) plant under the same developmental stage and growth conditions.

According to some embodiments of the invention, the partially or fully genomically multiplied plant has a dry matter weight similar to that of the tetraploid Durum wheat (Triticum durum) plant under the same developmental stage and growth conditions.

According to some embodiments of the invention, the partially or fully genomically multiplied plant has an average plant height similar to that of the tetraploid Durum wheat (Triticum durum) plant under the same developmental stage and growth conditions.

According to some embodiments of the invention, the partially or fully genomically multiplied plant has a seed number per spike at least as similar to that of the tetraploid Durum wheat (Triticum durum) plant under the same developmental stage and growth conditions.

According to some embodiments of the invention, the fertility is determined by at least one of:

number of seeds per plant;

seed set assay;

gamete fertility assay; and

acetocarmine pollen staining.

According to some embodiments of the invention, the partially or fully genomically multiplied plant is a hexaploid.

According to some embodiments of the invention, the partially or fully genomically multiplied plant is an octaploid.

According to some embodiments of the invention, the partially or fully genomically multiplied plant is capable of cross-breeding with a hexaploid wheat.

According to some embodiments of the invention, the hexaploid wheat is a bread wheat (Triticum aestivum L.).

According to an aspect of some embodiments of the present invention there is provided a plant part of the partially or fully genomically multiplied Durum wheat plant.

According to an aspect of some embodiments of the present invention there is provided a processed product of the partially or fully genomically multiplied plant or plant part.

According to some embodiments of the invention, the processed product is selected from the group consisting of food, feed, construction material and biofuel.

According to some embodiments of the invention, the food or feed is selected from the group consisting of extruded or non-extruded pasta, macaroni products, couscous, bulgur, Frekeh, breakfast cereals, bread, desserts, poultry and livestock feed.

According to an aspect of some embodiments of the present invention there is provided a meal produced from the partially or fully genomically multiplied plant or plant part.

According to some embodiments of the invention, the partially or fully genomically multiplied plant part is a seed or grain (may be interchangeably used herein).

According to an aspect of some embodiments of the present invention there is provided an isolated regenerable cell of the partially or fully genomically multiplied Durum wheat plant.

According to some embodiments of the invention, the cell exhibits genomic stability for at least 5 passages in culture.

According to some embodiments of the invention, the cell is from a mertistem, a pollen, a leaf, a root, a root tip, an anther, a pistil, a flower, a seed, grain, a straw or a stem.

According to an aspect of some embodiments of the present invention there is provided a tissue culture comprising the regenerable cells.

According to an aspect of some embodiments of the present invention there is provided a method of producing Durum wheat seeds, comprising self-breeding or cross-breeding the partially or fully genomically multiplied plant.

According to an aspect of some embodiments of the present invention there is provided a method of developing a hybrid plant using plant breeding techniques, the method comprising using the partially or fully genomically multiplied plant as a source of breeding material for self-breeding and/or cross-breeding.

According to an aspect of some embodiments of the present invention there is provided a method of producing Durum wheat meal, the method comprising:
(a) harvesting grains of the partially or fully genomically multiplied Durum plant or plant part; and
(b) processing the grains so as to produce the Durum meal.

According to an aspect of some embodiments of the present invention there is provided a method of generating a Durum wheat seed having a partially or fully multiplied genome, the method comprising contacting the Durum wheat (Triticum durum) seed with a G2/M cell cycle inhibitor under a transiently applied magnetic field thereby generating the Durum wheat seed having a partially or fully multiplied genome.

According to some embodiments of the invention, the G2/M cell cycle inhibitor comprises a microtubule polymerization inhibitor.

According to some embodiments of the invention, the microtubule polymerization inhibitor is selected from the group consisting of colchicine, nocodazole, oryzaline, trifluraline, vinblastine sulphate and analogs of each.

According to some embodiments of the invention, the method further comprises sonicating the seed prior to the contacting.

According to some embodiments of the invention, the method further comprises contacting the seed with a DNA protectant.

According to some embodiments of the invention, the DNA protectant is selected from the group of an antioxidant and a histone.

According to an aspect of some embodiments of the present invention there is provided a sample of representative seeds of a Durum wheat plant having a partially or fully multiplied genome being at least as fertile as a tetraploid Durum wheat (Triticum durum) plant isogenic to said genomically multiplied Durum wheat plant when grown under the same conditions and being of the same developmental stage, wherein said sample has been deposited under the Budapest Treaty at the NCIMB under NCIMB 42002.

According to an aspect of some embodiments of the present invention there is provided a sample of representative seeds of a Durum wheat plant having a partially or fully multiplied genome being at least as fertile as a tetraploid Durum wheat (Triticum durum) plant isogenic to said genomically multiplied Durum wheat plant when grown under the same conditions and being of the same developmental stage, wherein said sample of said Durum wheat plant having said partially or fully multiplied genome has been deposited under the Budapest Treaty at the NCIMB under NCIMB 42002.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-F are images of spikes and grains of genomically multiplied Durum wheat plants as compared to their isogenic tetraploid progenitors;

FIGS. 2A-C are images of a tetraploid Durum wheat (line E-2009-1, FIG. 2A), genomically multiplied hexaploid Durum wheat female plant (D3, FIG. 2B), and a hybrid plant (FIG. 2C) generated by crossing the female hexaploid plant with a male bread wheat line.

### DESCRIPTION OF SPECIFIC EMBODIMENTS OF THE INVENTION

The present invention, in some embodiments thereof, relates to Durum wheat plants having a partially or fully multiplied genome and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Induced polyploidy has been suggested for increasing plant yields. To date, however, induced polyploidy has been successfully achieved for only a few plant species.

The present inventors have now designed a novel procedure for induced genome multiplication in Durum wheat that results in plants which are genomically stable and fertile. The induced polyploid plants are devoid of undesired genomic mutations and are characterized by larger and heavier grains, higher spikelet number and length, and thus are considered of higher vigor and yield than that of the isogenic progenitor plant having a tetraploid genome (see Table 3, below). These new traits may contribute to better climate adaptability and higher tolerance to biotic and abiotic stress. Furthermore, hybrid wheat seeds generated by pollen sterilization using the induced polyploid plants of the present invention may increase global wheat yield due to heterosis expression. In addition, the induced polyploid plant of some embodiments of the invention exhibits comparable or better fertility to that of the isogenic tetraploid progenitor plant already from early generations (e.g., first, second, third or fourth) following genome multiplication, negating the need for further breeding in order to improve fertility.

Thus, according to an aspect of the invention there is provided a Durum wheat plant having a partially or fully multiplied genome being at least as fertile as a tetraploid Durum wheat (Triticum durum) plant isogenic to said genomically multiplied Durum wheat plant when grown under the same conditions and being of the same developmental stage.

As used herein, the term “Durum wheat” (also referred to herein as “macaroni wheat”, “Triticum durum” or “Triticum turgidum durum”) refers to the Triticum durum species of the Triticum genus. Durum wheat is a tetraploid wheat, having twenty-eight chromosomes, The composition of the euploid (tetraploid, non-multiplied plant) is 4n=28 originating through intergeneric hybridization and polyploidization involving two diploid grass species: T. urartu (2=2x=14, AA genome) and a B-genome diploid related to Aegilops speltoides (2n=2x=14, SS genome) and is thus an allotetraploid species.

According to a specific embodiment the Durum wheat may be naturally occurring or a synthetic wheat.

Common varieties of Durum wheat that can be used as a source for genomic multiplication include, but are not limited to: Divide 2005, Grenora 2005, Alkabo 2005, Dilse 2002, Pierce 2001, Lebsock 1999, Plaza 1999, Maier 1998, Mountrail 1998, Belzer 1997, Ben 1996 and Munich 1995.

“A plant” refers to a whole plant or portions thereof (e.g., seeds, stems, fruit, leaves, flowers, tissues, straw, etc.), processed or non-processed [e.g., seeds, meal (semolina), dry tissue, cake etc.], regenerable tissue culture or cells isolated therefrom.

According to some embodiments, the term plant as used herein also refers to hybrids having one of the induced polyploid plants as at least one of its ancestors, as will be further defined and explained hereinbelow.

As used herein “partially or fully multiplied genome” refers to an addition of at least one chromosome, an ancestral genome set (e.g., AA, BB), a mixed ancestral set of chromosomes (e.g., AB) that result in a hexaploid plant or a full multiplication of the genome that results in an octaploid plant (8N) or more.

The genomically multiplied plant of the invention is also referred to herein as “induced polyploid” plant.

According to a specific embodiment, the induced polyploid plant is 4N.

According to a specific embodiment, the induced polyploid plant is 5N.

According to a specific embodiment, the induced polyploid plant is 6N.

According to a specific embodiment, the induced polyploid plant is 7N.

According to a specific embodiment, the induced polyploid plant is 8N.

According to a specific embodiment, the induced polyploid plant is 9N.

According to a specific embodiment, the induced polyploid plant is 10N.

According to a specific embodiment, the induced polyploid plant is 11N.

According to a specific embodiment, the induced polyploid plant is 12N.

According to a specific embodiment, the induced polyploid plant is not a genomically multiplied haploid plant.

As mentioned, the induced polyploid is at least as fertile as the tetraploid Durum wheat progenitor plant isogenic to the genomically multiplied Durum wheat when grown under the same (identical) conditions and being of the same (identical) developmental stage.

As used herein the term “fertile” refers to the ability to reproduce sexually. Fertility can be assayed using methods which are well known in the art. Alternatively, fertility is defined as the ability to set seeds. The following parameters may be assayed in order to determine fertility: the number of seeds (grains); seed set assay; gamete fertility may be determined by pollen germination such as on a sucrose substrate; and alternatively or additionally acetocarmine staining, whereby a fertile pollen is stained.

As used herein the term “stable” or “genomic stability” refers to the number of chromosomes or chromosome copies, which remains constant through several generations, while the plant exhibits no substantial decline in at least one of the following parameters: yield, fertility, biomass and vigor

According to a specific embodiment, stability is defined as producing a true to type offspring, keeping the variety strong and consistent.

According to an embodiment of the invention, the genomically multiplied plant is isogenic to the source plant, namely the tetraploid Durum plant. The genomically multiplied plant has substantially the same genomic composition as the diploid plant in quality but not in quantity.

According to a specific embodiment, the plant exhibits genomic stability for at least 2, 3, 5, 10 or more passages in culture or generations of a whole plant.

According to some embodiments of the present invention, a mature genomically multiplied plant has at least about the same (+/−10%, 20% or 30%) number of seeds as it's isogenic tetraploid progenitor grown under the same conditions; alternatively or additionally the genomically multiplied plant has at least 90% fertile pollen that are stained by acetocarmine; and alternatively or additionally at least 90% of seeds germinate on sucrose. The hexaploid or octaploid plants generated according to the present teachings have total yield/plant which is higher by at least 5%, 10%, 15%, 20% or 25% than that of the isogenic progenitor plant. For example 5-10%, 1-10%, 10-20% 10-100% or 50-150% higher yield than that of the isogenic tetraploid plant grown under the same conditions and being of the same developmental stage. According to a specific embodiment, yield is measured using the following formula:

Yield per plant=total grain number/plant×grain weight

Comparison assays done for characterizing traits (e.g., fertility, yield, biomass and vigor) of the genomically multiplied plants of the present invention are typically effected in comparison to it's isogenic progenitor (hereinafter, “the tetraploid progenitor plant”) when both are being of the same developmental stage and both are grown under the same growth conditions.

According to a specific embodiment, the genomically multiplied plant is characterized by a spike number at least as similar to that of the tetraploid Durum wheat (Triticum durum) isogenic progenitor plant of the same developmental stage and grown under the same growth conditions. According to a specific embodiment the spike number is higher by 2%, 3%, 4%, 5%, 6%, 7%, 8%, 9%, 10% or even more 15% or 20% (e.g., 2-20%, 10-20%).

According to a specific embodiment, the genomically multiplied plant is characterized by a spikelet number at least as similar to that of the tetraploid Durum wheat (Triticum durum) isogenic progenitor plant of the same developmental stage and grown under the same growth conditions. According to a specific embodiment the a spikelet number is higher by 2%, 3%, 4%, 5%, 6%, 7%, 8%, 9%, 10% or even more 15% or 20% (e.g., 2-20%, 10-20%).

According to a specific embodiment, the genomically multiplied plant is characterized by a spike length at least as similar to that of the tetraploid Durum wheat (Triticum durum) isogenic progenitor plant of the same developmental stage and grown under the same growth conditions. According to a specific embodiment the spike length is higher by 2%, 3%, 4%, 5%, 6%, 7%, 8%, 9%, 10% or even more 15% or 20%.

According to a specific embodiment, the genomically multiplied plant is characterized by grain number per spikelet at least as similar to that of the tetraploid Durum wheat (Triticum durum) isogenic progenitor plant of the same developmental stage and grown under the same growth conditions. According to a specific embodiment the grain number per spikelet is higher by 2%, 3%, 4%, 5%, 6%, 7%, 8% 9%, 10% or even more 15% or 20%.

According to a specific embodiment, the genomically multiplied plant is characterized by grain weight at least as similar to that of the tetraploid Durum wheat (Triticum durum) isogenic progenitor plant of the same developmental stage and grown under the same growth conditions. According to a specific embodiment the grain weight is higher by 2%, 3%, 4%, 5%, 6%, 7%, 8%, 9%, 10% or even more 15% or 20%.

According to a specific embodiment, the genomically multiplied plant is characterized by a total grain number per plant at least as similar to that of the tetraploid Durum wheat (Triticum durum) isogenic progenitor plant of the same developmental stage and grown under the same growth conditions. According to a specific embodiment the total grain number per plant is higher by 10%, 15%, 20%, 25%, 30% 35%, 40%, 45%, 50% or even more 80% or 90%.

According to a specific embodiment, the genomically multiplied plant is characterized by grain yield per plant at least as similar to that of the tetraploid Durum wheat (Triticum durum) isogenic progenitor plant of the same developmental stage and grown under the same growth conditions. According to a specific embodiment the grain yield per plant is higher by 10%, 15%, 20%, 25%, 30%, 35%, 40%, 45%, 50% or even more 80% or 90%.

According to a specific embodiment, the genomically multiplied plant is characterized by a rust tolerance at least as similar to that of the tetraploid Durum wheat (Triticum durum) isogenic progenitor plant of the same developmental stage and grown under the same growth conditions. According to a specific embodiment the rust tolerance is higher by 2%, 3%, 4%, 5%, 6%, 7%, 8%, 9%, 10% or even more 15%, 20%, 30% or 40%.

According to a specific embodiment, the genomically multiplied plant is characterized by grain protein content at least as similar to that of the tetraploid Durum isogenic progenitor plant of the same developmental stage and grown under the same growth conditions. According to a specific embodiment the grain protein content is higher or lower by about 0-20% of that of the isogenic progenitor plant of the same developmental stage and grown under the same growth conditions.

According to a specific embodiment, the genomically multiplied plant is characterized by grain yield per growth area at least as similar to that of the tetraploid Durum isogenic progenitor plant of the same developmental stage and grown under the same growth conditions. According to a specific embodiment the grain yield per growth area is higher by 10%, 15%, 20%, 25%, 30%, 35%, 40%, 45%, 50% or even more 80%, 90%, 100%, 200, %, 250%, 300%, 400% or 500%. According to a specific embodiment the grain yield per growth area is higher by 0.1-5, 0.3-5, 0.4-2.5, 1-5, 2-3 or 2-2.5 fold than that of the isogenic progenitor plant of the same developmental stage and grown under the same growth conditions.

According to a specific embodiment, the genomically multiplied plant is characterized by grain yield per plant at least as similar to that of the tetraploid Durum isogenic progenitor plant of the same developmental stage and grown under the same growth conditions. According to a specific embodiment the grain yield per plant is higher by 5%, 10%, 15%, 20%, 25%, 30%, 35%, 40%, 45%, 50% or even more 80% 90%, 100%, 200, %, 250%, 300%, 400% or 500%. According to a specific embodiment the grain yield per plant is higher by 0.1-5, 0.3-5, 0.4-2.5, 1-5, 2-3 or 2-2.5 fold than that of the isogenic progenitor plant of the same developmental stage and grown under the same growth conditions.

Interestingly, the plants of the invention are characterized by an above ground plant length (i.e., plant height) that is similar or even shorter than that of the isogenic progenitor plant of the same developmental stage and grown under the same growth conditions. According to a specific embodiment the plant length is shorter by 2%, 3%, 4%, 5%, 6%, 7%, 8%, 9% or even 10%.

Plants of the invention are characterized by at least one, two, three, four or all of higher biomass, yield, grain yield, grain yield per growth area, grain protein content, grain weight, stover yield, seed set, chromosome number, genomic composition, percent oil, vigor, insect resistance, pesticide resistance, drought tolerance, and abiotic stress tolerance than the tetraploid Durum plant isogenic thereto.

It will be appreciated that while a certain trait of the induced polyploid plant may be inferior with respect to the isogenic progenitor others can be superior thus providing an overall superior phenotype.

For example, the induced polyploid line or hybrid, may have a seed weight which is inferior with respect to the weight of the isogenic progenitor but seed weight/plant or growth area which is superior to that of the isogenic progenitor.

Likewise, the induced polyploid line or hybrid, may have a seed weight which is inferior with respect to the weight of the isogenic progenitor but protein content which is superior to that of the isogenic progenitor.

According to a specific embodiment, the plant is non-transgenic.

According to another embodiment, the plant is transgenic for instance by expressing a heterologous gene conferring pest resistance or morphological traits for cultivation. For example, the parent plant or the induced polyploid plant can express a transgene that is associated with improved nutritional value. For instance, Dx5 and Dy10 high-molecular-weight (HMW) glutenin subunits, have been associated with superior bread-making quality but are absent from Durum wheats.

Genomically multiplied plant seeds of the present invention can be generated using an improved method of colchicination, as described below.

Thus, according to an aspect of the invention, there is provided a method of generating a Durum wheat seed having a partially or fully multiplied genome, the method comprising contacting the Durum wheat (Triticum durum) seed with a G2/M cell cycle inhibitor under a transiently applied magnetic field, thereby generating the Durum wheat seed having a partially or fully multiplied genome.

Typically, the G2/M cycle inhibitor comprises a microtubule polymerization inhibitor.

Examples of microtubule cycle inhibitors include, but are not limited colchicine, colcemid, trifluralin, oryzalin, benzimidazole carbamates (e.g. nocodazole, oncodazole, mebendazole, R 17934, MBC), o-isopropyl N-phenyl carbamate, chloroisopropyl N-phenyl carbamate, amiprophos-methyl, taxol, vinblastine, griseofulvin, caffeine, bis-ANS, maytansine, vinbalstine, vinblastine sulphate, podophyllotoxin and analogs of each.

The G2/M inhibitor is comprised in a treatment solution which may include additional active ingredients such as antioxidants, detergents and histones that are used as DNA protectants.

As used herein a “DNA protectant” relates to a compound or a condition that allows DNA multiplication without jeopardizing the composition of the DNA (≦0.001% mutations).

While treating the seeds with a treatment solution which comprises the G2/M cycle inhibitor, the plant can be further subjected to a magnetic field of at least 700 gauss (e.g., 1350 Gauss) for about 2 hr. The seeds are placed in a magnetic field chamber such as that described in Example 1. After the indicated time, the seeds are removed from the magnetic field.

To improve permeability of the seeds to the treatment solution, the seeds can be subjected to ultrasound treatment (e.g., 40 KHz for 5 to 20 min) prior to the contacting with the G2/M cycle inhibitor.

Wet seeds may respond better to treatment and therefore seeds can be soaked in an aqueous solution (e.g., distilled water) at the initiation of treatment.

According to a specific embodiment, the entire treatment can be performed in the dark and at room temperature (about 23-26° C.) or lower [e.g., for the ultrasound (US) stage].

Thus according to a specific embodiment, the seeds can be soaked in water at room temperature and then subjected to US treatment in distilled water.

Once permeated, the seeds can be placed in a receptacle containing the treatment solution and a magnetic field in turned on. Exemplary ranges of G2/M cycle inhibitor concentrations are provided in Table 1, below. The treatment solution may further comprise DMSO, detergents, DNA protectants e.g., antioxidants and histones at the concentrations listed below.

Once the seeds removed from the magnetic field they can be subject to a second round of treatment with the G2/M cycle inhibitor. Finally, the seeds can be washed and seeded on appropriate growth beds. Optionally, the seedlings can be grown in the presence of Acadian™ (Acadian AgriTech) and Giberllon (the latter is used when treated with vinblastine, as the G2/M cycle inhibitor).

It will be appreciated that the above method may be implemented on the whole plant or plant part such as described herein and not necessarily restricted to seeds.

Using the above teachings, the present inventors have established genomically multiplied Durum wheat plants.

Once established, the plants of the present invention can be propagated sexually or asexually such as by using tissue culturing techniques.

As used herein the phrase “tissue culture” refers to plant cells or plant parts from which wheat grass can be generated, including plant protoplasts, plant cali, plant clumps, and plant cells that are intact in plants, or part of plants, such as seeds, leaves, stems, pollens, straw, roots, root tips, anthers, ovules, petals, flowers, embryos, fibers and bolls.

According to some embodiments of the present invention, the cultured cells exhibit genomic stability for at least 2, 3, 4, 5, 7, 9 or 10 passages in culture.

Techniques of generating plant tissue culture and regenerating plants from tissue culture are well known in the art. For example, such techniques are set forth by Vasil., 1984. Cell Culture and Somatic Cell Genetics of Plants, Vol I, II, III, Laboratory Procedures and Their Applications, Academic Press, New York; Green et al., 1987. Plant Tissue and Cell Culture, Academic Press, New York; Weissbach and Weissbach. 1989. Methods for Plant Molecular Biology, Academic Press; Gelvin et al., 1990, Plant Molecular Biology Manual, Kluwer Academic Publishers; Evans et al., 1983, Handbook of Plant Cell Culture, MacMillian Publishing Company, New York; and Klee et al., 1987. Ann. Rev. of Plant Phys. 38:467 486.

The tissue culture can be generated from cells or protoplasts of a tissue selected from the group consisting of seeds, leaves, stems, pollens, roots, root tips, anthers, ovules, petals, flowers and embryos.

It will be appreciated that the plants of the present invention can also be used in plant breeding along with other wheat plants (i.e., self-breeding or cross breeding) in order to generate novel plants or plant lines which exhibit at least some of the characteristics of the Durum wheat plants of the present invention.

Plants resultant from crossing any of these with another plant can be utilized in pedigree breeding, transformation and/or backcrossing to generate additional cultivars which exhibit the characteristics of the genomically multiplied plants of the present invention and any other desired traits. Screening techniques employing molecular or biochemical procedures well known in the art can be used to ensure that the important commercial characteristics sought after are preserved in each breeding generation.

The goal of backcrossing is to alter or substitute a single trait or characteristic in a recurrent parental line. To accomplish this, a single gene of the recurrent parental line is substituted or supplemented with the desired gene from the nonrecurrent line, while retaining essentially all of the rest of the desired genes, and therefore the desired physiological and morphological constitution of the original line. The choice of the particular nonrecurrent parent will depend on the purpose of the backcross. One of the major purposes is to add some commercially desirable, agronomically important trait to the plant. The exact backcrossing protocol will depend on the characteristic or trait being altered or added to determine an appropriate testing protocol. Although backcrossing methods are simplified when the characteristic being transferred is a dominant allele, a recessive allele may also be transferred. In this instance, it may be necessary to introduce a test of the progeny to determine if the desired characteristic has been successfully transferred. Likewise, transgenes can be introduced into the plant using any of a variety of established transformation methods well-known to persons skilled in the art, such as: Gadaleta et al. J. Cereal Science2008 43:435-445; Gressel., 1985. Biotechnologically Conferring Herbicide Resistance in Crops: The Present Realities, In: Molecular Form and Function of the plant Genome, L van Vloten-Doting, (ed.), Plenum Press, New York; Huftner, S. L., et al., 1992, Revising Oversight of Genetically Modified Plants, Bio/Technology; Klee, H., et al., 1989, Plant Gene Vectors and Genetic Transformation: Plant Transformation Systems Based on the use of Agrobacterium tumefaciens, Cell Culture and Somatic Cell Genetics of Plants; and Koncz, C., et al. 1986, Molecular and General Genetics.

Using the present teachings, the present inventors were able to generate a number of plant varieties which are induced polyploids. A sample of representative seeds of, wherein a sample of the Durum wheat has been deposited under the Budapest Treaty at the NCIMB under NCIMB 42002 on Jul. 4, 2012. The NCIMB 42002 corresponds to the induced polyploidE-EP-V 1.

It will be appreciated that plants or hybrid plants of the present invention can be genetically modified such as in order to introduce traits of interest e.g. enhanced resistance to stress (e.g., biotic or abiotic).

Thus, the present invention provides novel genomically multiplied plants and cultivars, and seeds and tissue culture for generating same.

The plant of the present invention is capable of self-breeding or cross-breeding with a hexaploid wheat [e.g., common wheat (Triticum aestivum L.)], or other wheat species or wheat of various ploidies (e.g,. induced high-ploidy wheat as described herein).

Such hybrids have been generated i.e., common wheat crossed with induced polyploid Durum wheat were generated by the present inventors and are shown in Example 6.

Thus, such hybrids plants exhibit grain size similar to that of the tetraploid Durum wheat (Triticum durum) plant (±5-20%) under the same developmental stage and growth conditions. According to some embodiments, the grain size is higher (+5-20%) in the hybrid than that of the tetraploid isogenic plant. According to some embodiments, the grain size is lower (+5-20%) in the hybrid than that of the tetraploid isogenic plant.

Alternatively or additionally, such hybrid plants have grain protein content similar to that of said tetraploid Durum wheat (Triticum durum) plant (±5-20%) under the same developmental stage and growth conditions. According to some embodiments, the grain protein content is higher (+5-20%) in the hybrid than that of the tetraploid isogenic plant. According to some embodiments, the grain protein content is lower (+5-20%) in the hybrid than that of the tetraploid isogenic plant.

Alternatively or additionally, such hybrid plants have a dry matter weight similar to that of said tetraploid Durum wheat (Triticum durum) plant (±5-20%) under the same developmental stage and growth conditions. According to some embodiments, the dry matter weight is higher (+5-20%) in the hybrid than that of the tetraploid isogenic plant. According to some embodiments, the dry matter weight is lower (+5-20%) in the hybrid than that of the tetraploid isogenic plant.

Alternatively or additionally, such hybrid plants are as high (above ground) as said tetraploid Durum wheat (Triticum durum) plant (±5-20%) under the same developmental stage and growth conditions. According to some embodiments, plant height is higher (+5-20%) in the hybrid than that of the tetraploid isogenic plant. According to some embodiments, the plant height is lower (+5-20%) in the hybrid than that of the tetraploid isogenic plant.

Alternatively or additionally, such hybrid plants have a seed number per spike or a spike width or seeds/spike at least as similar to that of said tetraploid Durum wheat (Triticum durum) plant (±5-20%) under the same developmental stage and growth conditions. According to some embodiments the seed number per spike or spike width or seeds/spike is the same as in the tetraploid Durum wheat (Triticum durum) plant. According to some embodiments the seed number per spike or spike width or seeds/spike is the lower than (-5-20%) in the tetraploid Durum wheat (Triticum durum) plant. According to some embodiments the seed number per spike or spike width or seeds/spike is the higher than (+5-20%) as in the tetraploid Durum wheat (Triticum durum) plant.

Thus, the present invention further provides for a hybrid plant having as a parental ancestor the genomically multiplied plant as described herein.

For instance, the male parent may be the genomically multiplied plant while the female parent may be a tetraploid Durum wheat or a hexaploid common wheat. Alternatively, two induced genomically multiplied plants of the same (e.g., 6N×6N, 8N×8N) or different ploidy (e.g., 6N×8N) can be crossed.

According to a specific embodiment the invention provides for a hybrid Durum wheat plant having a partially or fully multiplied genome.

The present invention further provides for a seed bag which comprises at least 10%, 20% 50% or 100% of the seeds of the plants or hybrid plants of the invention.

The present invention further provides for a planted field which comprises any of the plants or hybrid plants of the invention.

Grains of the present invention are processed as meal used as supplements in foods or feed (e.g,. poultry and livestock).

Accordingly, the present invention further provides for a method of producing Durum wheat meal (e.g., semolina), the method comprising harvesting grains of the plant or hybrid plant of the invention; and processing the grains so as to produce meal.

Semolina, Durum granular, and Durum flour milled from Durum wheat are used to manufacture paste (extruded or non-extruded) and non-paste food products. Paste products are manufactured by mixing water with semolina or Durum flour to form unleavened dough, which is formed into different shapes and either cooked and eaten or dried for later consumption. Pasta and couscous are examples of paste products (other examples are provided hereinbelow). Products of Durum wheat in a high moisture leavened or unleavened bread and cooked or steamed bulgur (cracked Durum wheat) and frekeh (parched immature wheat kernel) are non-paste food products.

Pasta Products

Italians categorize pasta into four main groups: long goods (spaghetti, vermicelli, and linguine), short goods (elbow macaroni, rigatoni, and ziti), egg noodles (pasta made with eggs), and specialty items (lasagna, manicotti, jumbo shells, and stuffed pasta. Italian extruded food and Oriental noodles differ. Pasta noodles are made from Durum or non-Durum wheat with a minimum requirement of 5.5% egg solids. Oriental noodles are made from non-Durum wheat flour.

In the Western Hemisphere and Europe, macaroni products are usually referred to as alimentary pastes. Macaroni (hollow tubes), spaghetti (solid rods), noodles (strips, either flat or oval), and shapes (stamped in various forms from sheets of dough) are known as the macaroni products.

Couscous

Couscous, a paste product made from mixing semolina with water, is considered one of the major food staples in North African countries, such as Egypt, Libya, Tunisia, Algeria, and Morocco. An estimated 10% of Durum wheat in the Near East is used to manufacture couscous.

Bulgur

Bulgur, a non-paste parboiled Durum wheat product, is one of the oldest cereal-based foods. Bulgur is used as a main dish or as one of the ingredients in most food consumed in Turkey, Syria, Jordan, Lebanon, and Egypt.

Bulgur making involves three steps: 1) The wheat is cleaned, soaked in water, and cooked to gelatinize the starch. 2) The cooked grain is cooled, dried, moistened, peeled to remove the bran (optional), redried, and cleaned by winnowing. 3) The grain is milled and sieved into three or four size grades: coarse, fine, very fine, and flour.

Frekeh or Firik

Frekeh is also known as firik. Frekeh, a non-paste Durum wheat product, is a staple food in North Africa and the Middle East, especially Syria. Frekeh is a parched green wheat that is used in the same way as rice, bulgur, and couscous.

The best frekeh is made from the largest, hardest, and greenest grains. Therefore, durum wheat, especially cultivars with large kernels, is the most suitable wheat for making frekeh. When processed from wheat harvested in late-milk to mid-dough stages, roughly 13 to 16 d after anthesis, frekeh is more delicious than that processed at the full-ripe stage, probably due to the higher contents of free simple sugars. Kernels in the early stages of development have high concentrations of minerals and vitamins, particularly thiamin and riboflavin.

Durum Wheat Breakfast Cereals

In the Middle East, mamuneih made from semolina cooked in water with butter and sugar is consumed as a hot breakfast cereal. In North America, large kernels of durum wheat are used to make a puffed durum wheat ready-to-eat breakfast cereal.

Durum Wheat Bread

Durum wheat is used to a larger extent in bread production in the Near East, Middle East, and Italy than in other parts of the world. In some Middle Eastern countries, 70 to 90% of Durum wheat is used for bread. Several types of bread are made from Durum wheat. Two-layered bread, khobz, is the most popular bread in Syria, Lebanon, and Jordan. In Egypt, two-layered bread is called baladi and shami. Single-layer bread also is popular, including tannur and saaj (Syria and Lebanon), Mountain bread and markouk (Lebanon), and mehrahrah. In Turkey, flat bread, tandir ekmegi, is made from Durum wheat. Thirty percent and 18% of Durum wheat in the Near East is used to make two-layered and single-layer breads, respectively.

Several kinds of bread are made in Italy from Durum wheat, depending on the shape of the bread and the region of the country. The common breads include fresedde in the province of Bari, frasella in the province of Foggia, and frasedda, frisedda, and frisa in the province of Salerno. A round, flat bread, cafone, is produced in Bari. A wheel-shaped Durum wheat bread, rote, is produced in the Bari and Foggia provinces. Sckanate is a large Durum bread typically made in Minervino, Altamura, Bitonto, and Gargano.

Desserts

In the Middle East, several desserts are made from semolina. Deep-fried semolina dough (mushabak), baked semolina dough (hariseh), and baked semolina mixture with vegetable oil, sugar, and nuts (halva) are common desserts in Syria, Lebanon, and Jordan. In Germany, kugel is a sweet noodle pudding that is used as a dessert and now is being marketed in North America.

Wheat grass is highly fermentable, which makes the plants or hybrids of the invention a good alternative for use in beer and other alcoholic beverages production and also useful for production of biofuels. Plants or hybrids of the invention can also be used in construction, such as a thatch for roofing.

It is expected that during the life of a patent maturing from this application many relevant DNA protectants, Durum wheat varieties, Durum wheat products and uses will be developed and the scope of the terms provided herein is intended to include all such new technologies a priori.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization - A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Genome Multiplication of Durum Wheat

Experimental Procedures

All stages were performed in the dark.

Seeds were soaked in a vessel full of water at about 25° C. for about 2 hr.

The seeds were transferred into a clean net bag and put into a distilled water-filled ultrasonic bath at about 23 to about 26° C. Sonication was applied (about 40 KHz) for about 5 to about 20 minutes. Temperature was kept below 26° C. The seeds bag was placed in a vessel containing the treatment solution (described below) at about 25° C. The vessel was placed within the magnetic field chamber (described below) and incubated for about 2 hr. Seeds were removed from the bag and placed on top of a paper towel bed on a plastic tray. A second layer of paper towel soaked with treatment solution was used as a cover. The seeds were incubated for about 12- about 48 hr at about 25° C. and kept wet for the whole incubation period. The seeds were collected into a clean vessel and washed with water (pH=7). A seedling tray of soil supplemented with about 25 ppm of 20:20:20 Micro Elements Fertilizer was prepared. Treated seeds were seeded to the tray and moved to nursery using a day temperature range of about 20- about 25° C., night temperature range of about 10- about 17° C. and minimal moisture of about 40%.

When using Vinblastine, 0.5-1.5% GIBERLLON were applied immediately after seeding. The seeds were treated with ACADIAN™ twice a week for the following 3 weeks.

### Treatment Solution:

DMSO 0.5%
TritonX100 5 drops/L
microtubule polymerization inhibitor
Antioxidant
Histones 50-100 μg/ml
pH=6
   Prepared in softened, nitrogen free water
   *for immediate use.

TABLE 1

| | Concentration | |
|---|---|---|
| Microtubule polymerization inhibitor | | |
| Vinblastine sulphate | 0.05-0.2% | |
| Colchicine | 0.1-0.5 | mg/ml |
| Nocodazole | 0.1-0.9% | |
| Oryzalin | 0.002-0.005% | |
| Trifluralin | 0.002-0.005% | |
| Antioxidants | | |
| Cyanidin 3-O-b-glucopyranoside | 25-100 | μg/ml |
| Baicalein | 10⁻⁶⁻10⁻⁴M | |
| Quercetin | 10⁻⁶⁻10⁻⁴M | |
| Trolox | 5-10 | mM |

Magnetic Field Details:

The magnetic field chamber consisted of two magnet boards located 11 cm from each other. The magnetic field formed by the two magnets is a coil-shaped magnetic field with a minimal strength of 1350 gauss in its central axis. The seeds were placed in a net bag within a stainless steel bath filled with treatment solution (as described above), and the bath was inserted into the magnetic chamber.

### Example 2

### Assessment of Ploidy Level by FACS

Table 2 below shows the DNA content in arbitrary units as assayed by FACS. First, the ploidy level was determined for a diploid, tetraploid (Durum) and hexaploid (bread) wheat. Then, the base line of the tetraploid wheat was set to 300. The ploidy of the multiplied lines is indicated in the Table. Evidently, both fully multiplied (8N) and partially multiplied (6N) plants were obtained.

TABLE 2

| | | | | Ploidy | |
|---|---|---|---|---|---|
| Code | Name | Generation | FACS Results | Level | Comments |
| | Triticum | Wild type | 240 | 2n | |
| | monococcum | | | | |
| 31 | Triticum durum | F8+ | 420 | 4n | Durum Wheat |
| | Triticum aestivum- | F8+ | 680 | 6n | Bread Wheat |
| | Rip | | | | |
| | Triticum aestivum-H | F8+ | 700 | 6n | Bread Wheat |
| | Triticum aestivum- | F8+ | 720 | 6n | Bread Wheat |
| | BB | | | | |

After Screening different Ploidy levels of each Speacies (See above), The Tetra-ploidy Durum control
was set to 300.

| | | | | | |
|---|---|---|---|---|---|
| 31 | 4 Control | F8+ | 300 | 4n | Durum Wheat |
| 32 | 4(101)1 | D3 | 620 | 8n | Stable High Ploidy Durum Wheat |
| 33 | 4(103)1 | D3 | 580 | 8n | Stable High Ploidy Durum Wheat |
| 34 | 4(103)2 | D3 | 620 | 8n | Stable High Ploidy Durum Wheat |
| 35 | 4(103)3 | D3 | 620 | 8n | Stable High Ploidy Durum Wheat |
| 36 | 4(105)1 | D3 | 600 | 8n | Stable High Ploidy Durum Wheat |
| 40 | 4(108)2 | D3 | 600 | 8n | Stable High Ploidy Durum Wheat |
| 41 | 4(108)3 | D3 | 640 | 8n | Stable High Ploidy Durum Wheat |
| 42 | 4(110)1 | D3 | 640 | 8n | Stable High Ploidy Durum Wheat |
| 43 | 4(111)1 | D3 | 660 | 8n | Stable High Ploidy Durum Wheat |
| 44 | 4(111)1-1 | D3 | 620 | 8n | Stable High Ploidy Durum Wheat |
| 45 | 4(112)1 | D3 | 600 | 8n | Stable High Ploidy Durum Wheat |
| 46 | 4(113)1 | D3 | 470 | 6n | Stable High Ploidy Durum Wheat |
| 47 | 4(113)2 | D3 | 640 | 8n | Stable High Ploidy Durum Wheat |
| 48 | 4(114)1 | D3 | 640 | 8n | Stable High Ploidy Durum Wheat |
| 49 | 4(114)2 | D3 | 580 | 8n | Stable High Ploidy Durum Wheat |

| Name | Generation | Ploidy Level | FACS Results | Comments |
|---|---|---|---|---|
| 4-Control | F6+ | 4n | 340 | Tetraploid Durum Wheat |
| 4-37 | D5 | EP | 500 | Stable High Ploidy Durum Wheat |

EP-stands for Enhanced polyploid or Induced Polyploid lines or Induced Polyploid Hybrid.
“4-Control” is the isogenic tetraploid lines used for genome multiplication. Each plant family are the self-seeds of different successfully genome multiplied inflorence.
“4-37” D5 indicates that the plants are fifth generation after genome multiplication procedure respectively.
In addition, D5 represents induced polyploid lines plants whose ploidy is higher than the isogenic source plan, as generated using the protocol of Example 1, above.

### Example 3

### Phenotypic Characterization of the Genomically Multiplied Durum Wheat

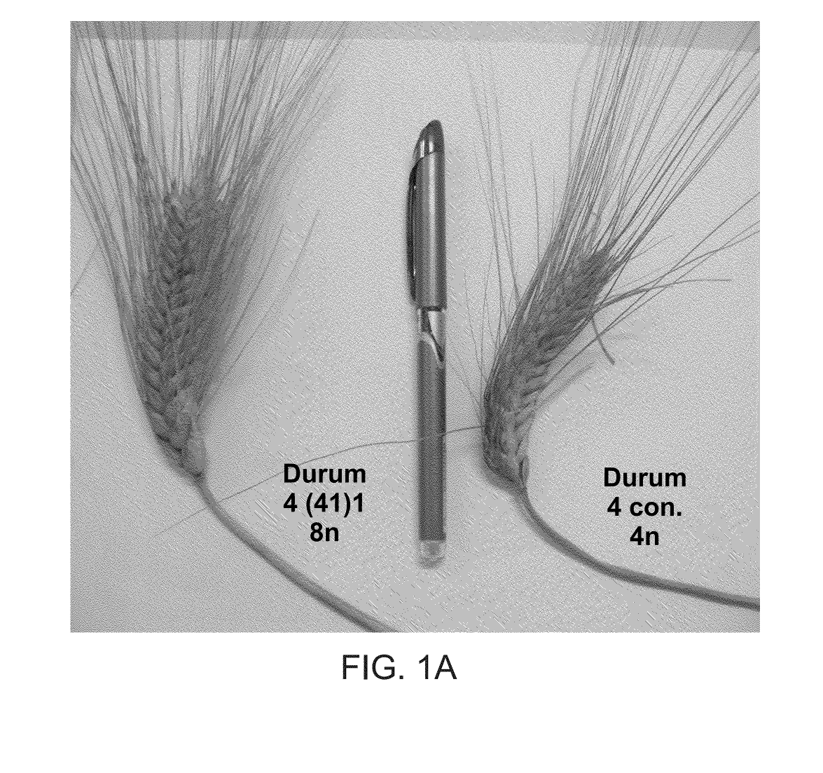

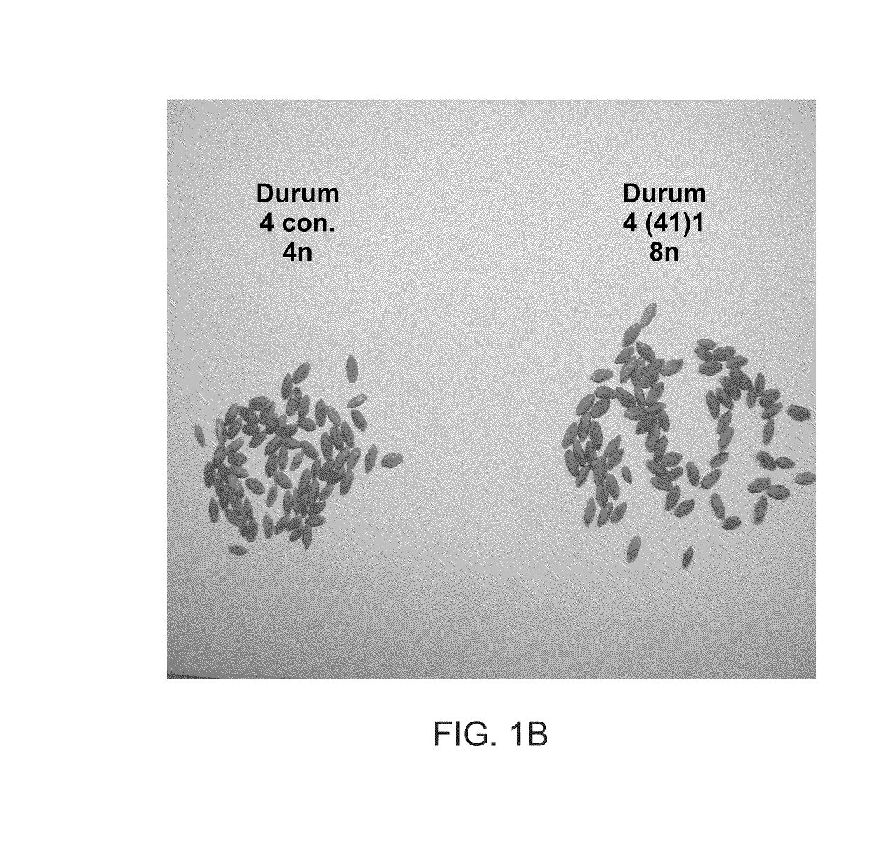

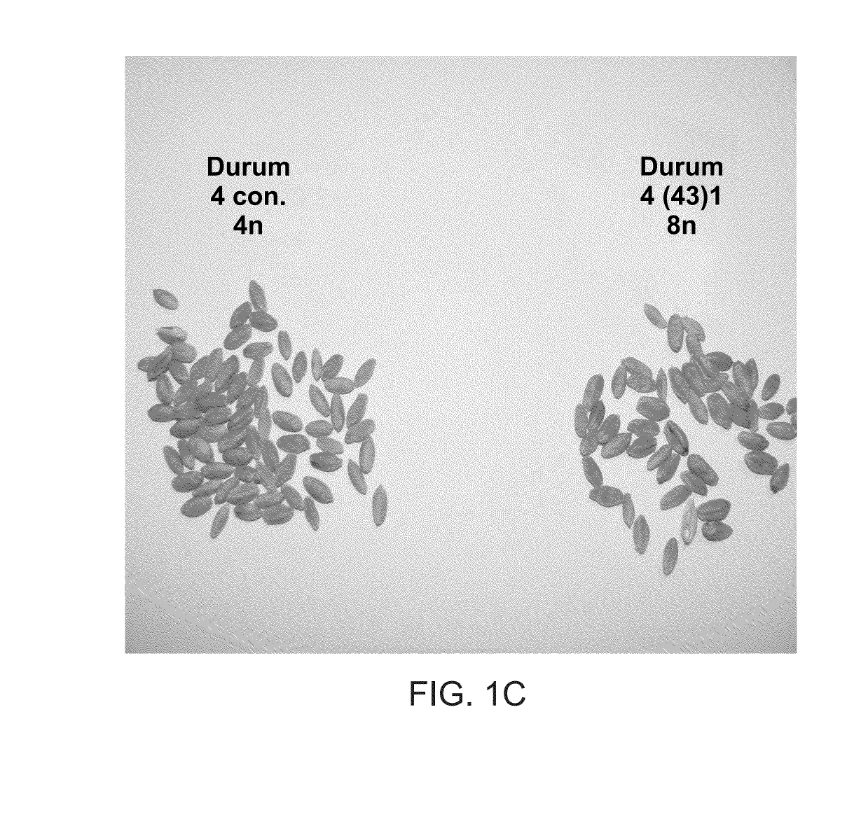

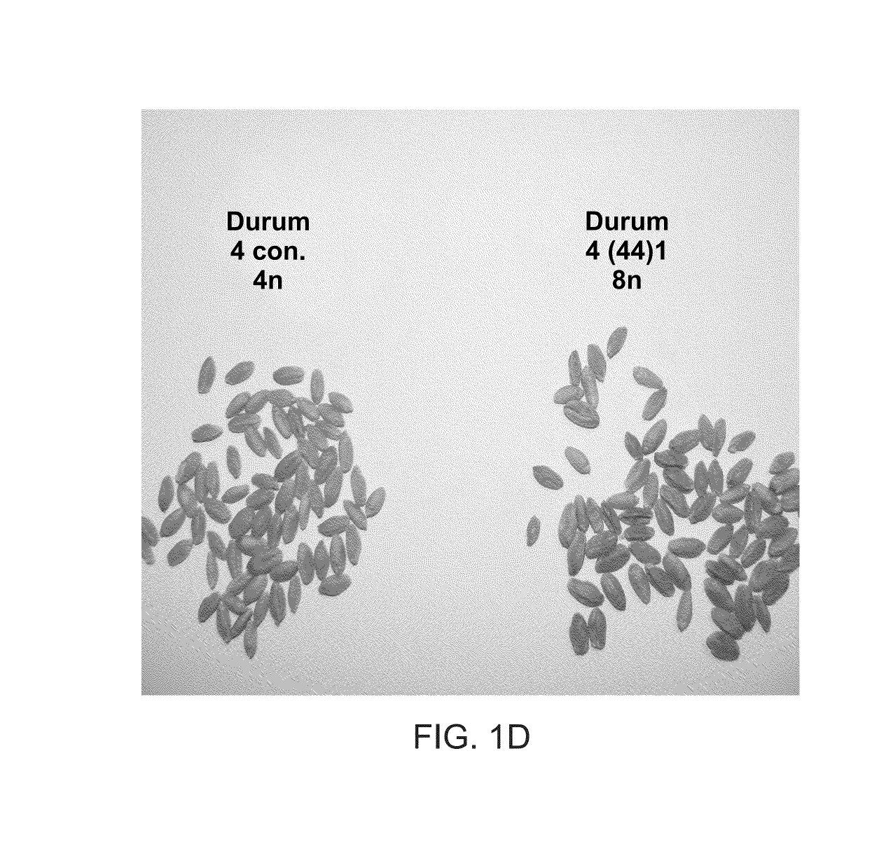

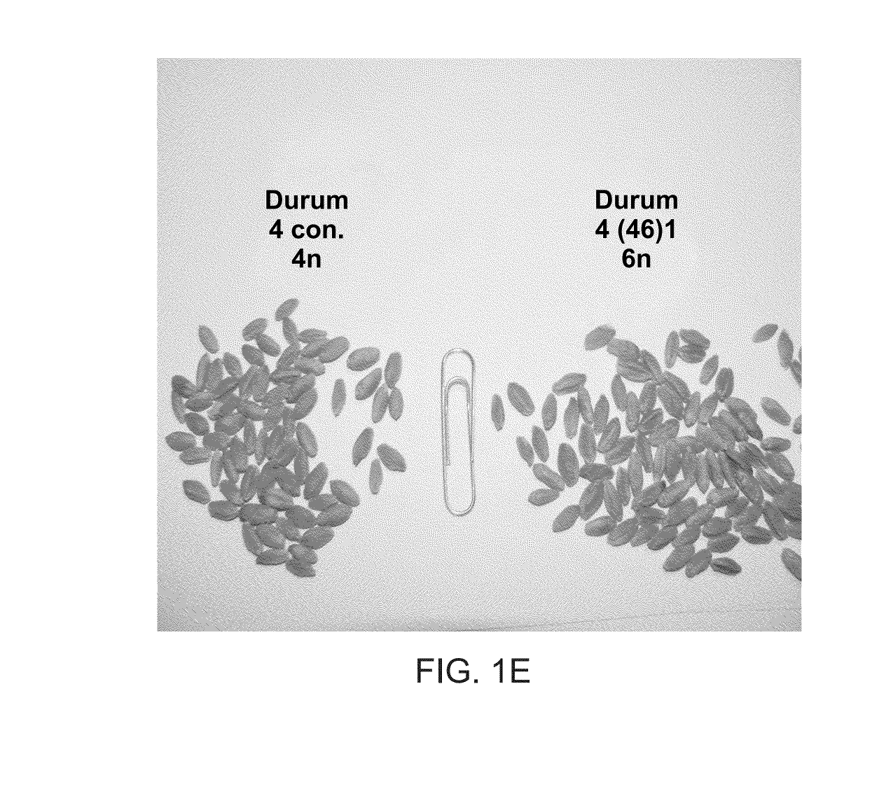

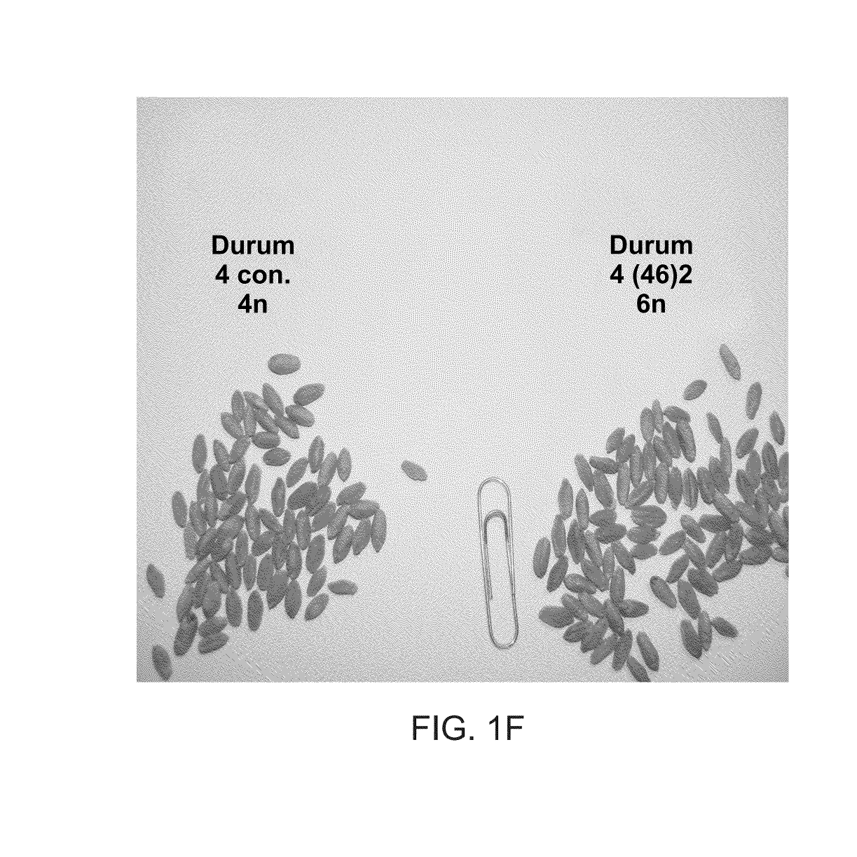

The fourth generation (D4) of the multiplied Durum wheat generated according to the teachings of Example 1 was subject to various phenotypic analyses, including thousand seeds weight, spike length, spike width and number of spikelets. The results are listed in Table 3 below. Representative pictures of the genomically multiplied plants are provided in FIGS. 1A-F.

TABLE 3

| | Gene- | Ploidy | 1000 seeds | Spikes's | Spike's | No. of |
|---|---|---|---|---|---|---|
| Name | ration | Level | weight | Length | Width | Spikelets |
| 4 | F8+ | 4n | 46.5 | 9 | 2 | 31 |
| Control | | | | | | |
| 4(41)1 | D4 | 8n | 58.3 | 11.5 | 2.4 | 31 |
| 4(43)1 | D4 | 8n | 47.8 | 10 | 2 | 31 |
| 4(44)1 | D4 | 8n | 54.1 | 11 | 2.2 | 31 |
| 4(46)1 | D4 | 6n | 45.8 | 9.5 | 2 | 31 |
| 4(46)2 | D4 | 6n | 48.6 | 10 | 2 | 31 |

### Example 4

### Generation of Hybrid Plants from 6N Durum Wheat and 6N Bread Wheat

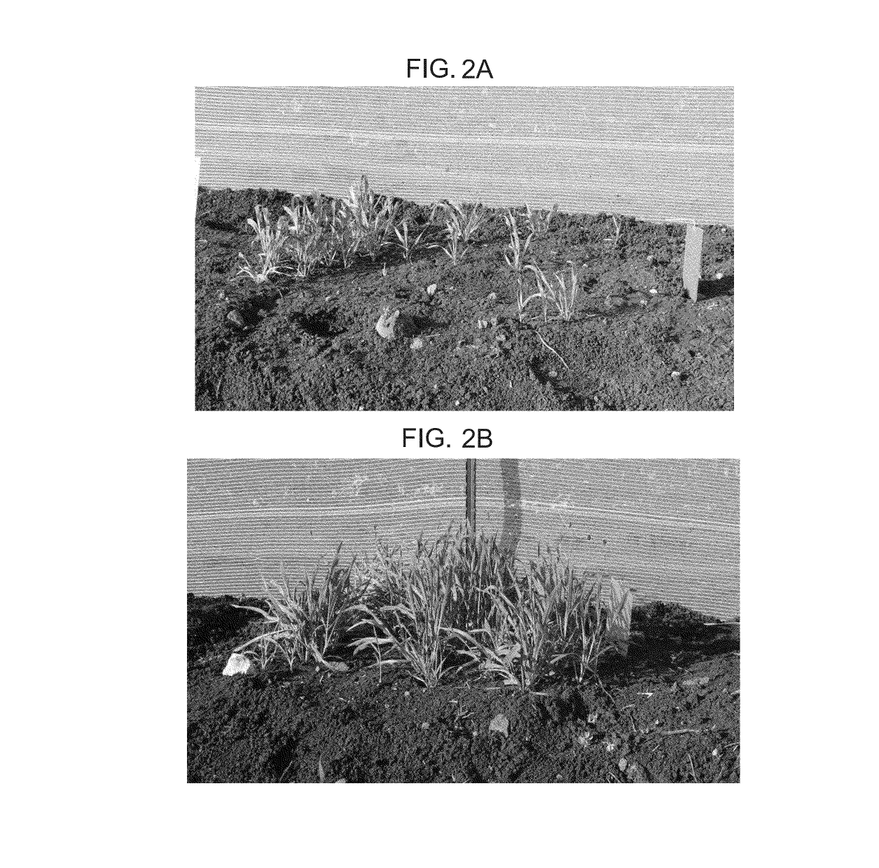

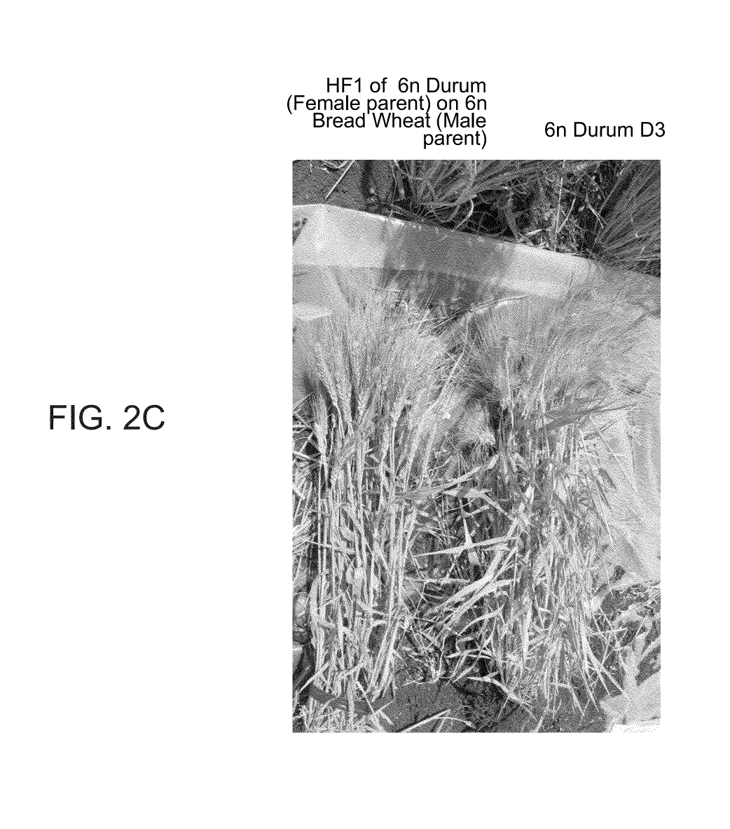

A hexaploid female Durum wheat line (4(37) was generated as described in Example 1 having the F8+as the isogenic tetraploid parent. The multiplied female line was crossed with the bread wheat male line, 2-2010(10)1, to give a hybrid plant designated, HF1W20(635)1. The hybrid exhibited superior traits as compared to the wild-type bread wheat, as evidenced by the number of spikes, spike length, number of spikeletes, grain weight, total weight and plant yield (see Table 4 below). Representative pictures of the hybrid plants are provided in FIGS. 2A-C.

TABLE 4

| | | | | | | total | | yield/plant | tolerance |
|---|---|---|---|---|---|---|---|---|---|
| | plant length | spike length | spikes | spiklets | grain/spiklets | grain/plant/100 | weght/1000 | 10 gr | to rust |
| | Units | | | | | | | | |
| Codes | cm | cm | # | # | # | # | grams | 10 grams | 1-sus′-5 resist′ |
| HF1 code: | 80 | 11 | 43 | 28 | 4 | 48.16 | 42 | 20.2272 | 3 |
| W20(635)1 | | | | | | | | | |
| Female | 80 | 11 | 52 | 30 | 4 | 62.4 | 44 | 27.456 | 3 |
| line: E-2009- | | | | | | | | | |
| 1D3 4(37) | | | | | | | | | |
| Male line: 2- | 70 | 10 | 40 | 27 | 3 | 32.4 | 40 | 12.96 | 3 |
| 2010(10)1 | | | | | | | | | |

### Example 5

### Phenotypic Characterization of Strips with Commercial Stand

Following basic preparation, the seeds as detailed below were pre-grown in a nursery and transplanted in strips with commercial stand. The experimental plots were irrigated by drip irrigation. Data was collected from four blocks of randomized replications. Plot size was 18.2 m². Sowing was at a density of 200 seeds/m². Harvest was done with a small grain experiment combine. Each plot was separately harvested into a new sack. The seeds were cleaned, after which weight and yield was measured.
“201-control”—Triticum Durum (spring Durum).
“207 EP”—Triticum Durum EP (spring Durum).
“208 EP”—Triticum Durum EP (spring Durum).

TABLE 5

| | | | Average | | Increase of | | |
|---|---|---|---|---|---|---|---|
| | | | Crop | | Yield Vs. | 1000 | Increase of |
| | | Ploidy | Yield | Standard | Control | Seeds | Yield Vs. |
| Line | Generation | Level | (Ton/Ha) | Deviation | (%) | Weight | Control (%) |
| 201- | F6+ | 4n | 6.0 | 0.4 | — | 37.7 | — |
| Control | | | | | | | |
| 207 | D5 | EP | 6.5 | 0.7 | 8.9 | 40.7 | 8.0 |
| 208 | D5 | EP | 6.5 | 0.5 | 8.6 | 41.5 | 10.1 |

Thus, all the tested grains of the polyploid lines Durum wheat plants having a partially or fully multiplied genome exhibited higher 1000 seeds weight compared to the grains of control plant under the same developmental stage and growth conditions. Seeds grain weight is one of the most important yield properties. These results support the high crop yield demonstrated in the present analysis. Indeed, the polyploid lines exhibited an increase in the crop yield of approximately nine percent compared to the control plant. Thus, the plants exhibited full seed set indicating that the induced polyploid (EP) plants had at least equivalent fertility as the control plants.

### Example 6

### Phenotypic Analysis of Single Polyploid Lines and Hybrids

Following basic preparation, the field cleared using herbicide and planted with the experimental plants, including control plants, the seeds were pre-grown in a nursery and transplanted in rows with inter-row spacing of 25 cm. Data was collected from 1-5 plants per line. The experimental plots were irrigated by drip irrigation. The seeds were cleaned, weight and the yield calculated.
“5-57-control”—Common wheat control (spring wheat), 6n.
“4-31-control”—Durum wheat (spring wheat), 4n.
“4-37-control”—Durum wheat EP (spring wheat).
843—Polyploid hybrid plant crossed from “5-57-control” (female spring common wheat, 6n)×“4-37-control” (male spring Durum wheat EP)
837 (reciprocal of 843)—Polyploid hybrid plant crossed from “4-37-control” (female spring Durum wheat EP) “5-57-control” (male spring common wheat, 6n).

TABLE 6

| Line/Hybrid Name | Cross | Species type |
|---|---|---|
| 5-57-control | | Common wheat (6n) |
| 4-31-control | | Durum wheat (4n) |
| 4-37-EP | | Polyploid Durum wheat |
| 843 | 5-57-control X 4-37-EP | Common wheat (6n) X |
| | | Durum wheat EP |
| 837 | 4-37-EP X 5-57-control | Common wheat (6n) X |
| | | Durum wheat EP |

TABLE 7
Plant Height of Polyploid Hybrids Plant Compared to Female Plant

| | Ploidy | |
|---|---|---|
| Name | Level | Plant Height |
| 5-57-control | 6n | 75 |
| 4-31-control | 6n | 92 |
| 4-37-EP | 6n | 90 |
| 843 | EP | 86 |
| 837 | EP | 90 |

Thus, the Durum wheat plant having a partially or fully multiplied genome exhibits essentially the same height as or higher height than the isogenic tetraploid control.

TABLE 8
Seed Weight of Polyploid Hybrids Plant
Compared to Control Plant

| | Ploidy | 1000 Seeds |
|---|---|---|
| Name | Level | Weight |
| 5-57-control | 6n | 53.2 |
| 4-31-control | 6n | 48.0 |
| 4-37-EP | 6n | 45.5 |
| 843 | EP | 45.0 |
| 837 | EP | 57.8 |

Thus all the tested grains of the polyploid hybrid Durum wheat plant having a partially or fully multiplied genome exhibited similar weight or lower weight compared to the grains of control plant under the same developmental stage and growth conditions. Seeds grain weight may be lower in the EP-line or hybrid than in the isogenic source, while the grain weight per plant or per growth area may be higher in the EP-line or hybrid than in the isogenic source. These results support the high crop yield demonstrated in the present analysis.

TABLE 9
Grain Protein Content of Polyploid Hybrids Plant
Compared to Control Plant

| | Ploidy | Grain Protein |
|---|---|---|
| Name | Level | Content |
| 5-57-control | 6n | 18.0 |
| 4-31-control | 6n | 17.6 |
| 4-37-EP | 6n | 18.5 |
| 843 | EP | 19.3 |
| 837 | EP | 20.7 |

Thus, the present results show that the polyploid hybrid plant grain protein content was 7%-15% higher compared to that of the common wheat control plant and 9.6%-15% higher compared to that of the Durum wheat control plant. In addition, the grain protein content of the EP Durum wheat plant was 5% higher than the Durum wheat control plant. Thus, the genome multiplication protocol affected grain protein content in the polyploid hybrids as well as the EP Durum wheat plants.

TABLE 10
Grain Weight of Polyploid Hybrids Plant Compared to Control Plant

| | | | | Grain Weight |
|---|---|---|---|---|
| | | Grain | Grain Weight per | per Plant |
| | | Weight | Plant | Over |
| | Ploidy | per Plant | Over | 4-31-Control |
| Name | Level | (gr) | 5-57-Control (%) | (%) |
| 5-57-control | 6n | 88.4 | | |
| 4-31-control | 6n | 160.8 | | |
| 4-37-EP | 6n | 171.2 | | 6.5 |
| 843 | EP | 164 | 85.5 | 2.0 |
| 837 | EP | 186 | 110.5 | 15.7 |

Thus, the polyploid hybrid Durum wheat plant having a partially or fully multiplied genome exhibited a significant increase in the grain weight indicating on the increase of crop yield of up to 110% compared to the common wheat control plant and up to 15.7% compared to the Durum wheat control plant under the same developmental stage and growth conditions. Thus, the plants exhibited full seed set indicating that the induced polyploid (EP) plants and hybrids had at least equivalent fertility as the control plants.

TABLE 11
Dry Matter Weight of Polyploid Hybrids Plant Compared to Control
Plant

| | | Dry Matter | Dry Matter Weight |
|---|---|---|---|
| | | Weight | per plant |
| | Ploidy | per plant | Over 5-57-Control |
| Name | Level | (Ton/Ha) | (%) |
| 5-57-control | 6n | 16.6 | |
| 4-31-control | 6n | 53.2 | |
| 4-37-EP | 6n | 48.1 | |
| 843 | EP | 32.6 | 96.4 |
| 837 | EP | 66.4 | 400 |

Thus, the polyploid hybrid Durum wheat plant having a partially or fully multiplied genome demonstrated an increase in dry matter weight of tens percent the control plant under the same developmental stage and growth conditions. Hence, the higher quantity of the dry matter weight is indicative of high bio-mass accumulation in the polyploid hybrid plants. In addition, these results indicate that the vigor and the heterosis effect are higher in the hybrid plants compared to control plants.

TABLE 12
Spike Data of Polyploid Hybrids Compared to Female Control Lines

| | | | No. Seeds per |
|---|---|---|---|
| Name | Spike length | Spike width | Spikelet |
| 5-57-control | 15.0 | 3.0 | 8.5 |
| 4-31-control | 7.5 | 2.5 | 4.5 |
| 4-37-EP | 10.0 | 3.0 | 3.5 |
| 843 | 18.5 | 2.2 | 7 |
| 837 | 15.0 | 2.0 | 5 |

Thus, the present results showed that the spike length and width were higher when comparing EP Durum wheat plant to Durum control wheat. All EP plants, EP lines as well as hybrids, demonstrated higher spike length and width compared to control Durum wheat. In conclusion, crossing of EP Durum wheat with common wheat plants resulted in higher spike length and width.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A Durum wheat plant having a partially or fully multiplied genome being at least as fertile as a tetraploid Durum wheat (Triticum durum) plant isogenic to said genomically multiplied Durum wheat plant when grown under the same conditions and being of the same developmental stage.

2. A hybrid plant having as a parental ancestor the plant of claim 1.

3. A hybrid Durum wheat plant having a partially or fully multiplied genome.

4. A planted field comprising the plant of claim 1.

5. A sown field comprising seeds of the plant of claim 1.

6. The plant of claim 1 being non-transgenic.

7. The plant of claim 1 having a spike number at least as similar to that of said tetraploid Durum wheat (Triticum durum) plant under the same developmental stage and growth conditions.

8. The plant of claim 1 having a spike width at least as similar to that of said tetraploid Durum wheat (Triticum durum) plant under the same developmental stage and growth conditions.

9. The plant of claim 1 having a spikelet number at least as similar to that of said tetraploid Durum wheat (Triticum durum) plant under the same developmental stage and growth conditions.

10. The plant of claim 1, having a spike length at least as similar to that of said tetraploid Durum wheat (Triticum durum) plant under the same developmental stage and growth conditions.

11. The plant of claim 1, having grain weight at least as similar to that of said tetraploid Durum wheat (Triticum durum) plant under the same developmental stage and growth conditions.

12. The plant of claim 1, having grain yield per area at least as similar to that of said tetraploid Durum wheat (Triticum durum) plant under the same developmental stage and growth conditions.

13. The plant of claim 1, having grain yield per plant at least as similar to that of said tetraploid Durum wheat (Triticum durum) plant under the same developmental stage and growth conditions.

14. The plant of claim 1, having grain size similar to that of said tetraploid Durum wheat (Triticum durum) plant under the same developmental stage and growth conditions.

15. The plant of claim 1, having grain protein content similar to that of said tetraploid Durum wheat (Triticum durum) plant under the same developmental stage and growth conditions.

16. The plant of claim 1, having a dry matter weight similar to that of said tetraploid Durum wheat (Triticum durum) plant under the same developmental stage and growth conditions.

17. The plant of claim 1, being as high as said tetraploid Durum wheat (Triticum durum) plant under the same developmental stage and growth conditions.

18. The plant of claim 1, having a seed number per spike at least as similar to that of said tetraploid Durum wheat (Triticum durum) plant under the same developmental stage and growth conditions.

19. The plant of claim 1, wherein said fertility is determined by at least one of:
number of seeds per plant;
gamete fertility assay; and
acetocarmine pollen staining.

20. The plant of claim 1, being a hexaploid.

21. The plant of claim 1, being an octaploid.

22. The plant of claim 1, capable of cross-breeding with a hexaploid wheat.

23. (canceled)

24. A plant part of the Durum wheat plant of claim 1.

25. A processed product of the plant or plant part of claim 1.

26. The processed product of claim 25, selected from the group consisting of food and feed.

27. (canceled)

28. A meal produced from the plant or plant part of claim 1.

29. The plant part of claim 24 being a seed.

30. An isolated regenerable cell of the Durum wheat plant of claim 1.

31. The cell of claim 29, exhibiting genomic stability for at least 5 passages in culture.

32-33. (canceled)

34. A method of producing Durum wheat seeds, comprising self-breeding or cross-breeding the plant of claim 1.

35. A method of developing a hybrid plant using plant breeding techniques, the method comprising using the plant of claim 1 as a source of breeding material for self-breeding and/or cross-breeding.

36. A method of producing Durum wheat meal, the method comprising:
(a) harvesting grains of the Durum plant or plant part of claim 1; and
(b) processing said grains so as to produce the Durum meal.

37. A method of generating a Durum wheat seed having a partially or fully multiplied genome, the method comprising contacting the Durum wheat (Triticum durum) seed with a G2/M cell cycle inhibitor under a transiently applied magnetic field thereby generating the Durum wheat seed having a partially or fully multiplied genome.

38. The method of claim 37, wherein said G2/M cell cycle inhibitor comprises a microtubule polymerization inhibitor.

39. The method of claim 38, wherein said microtubule polymerization inhibitor is selected from the group consisting of colchicine, nocodazole, oryzaline, trifluraline and vinblastine sulphate.

40. The method of claim 37, further comprising sonicating said seed prior to said contacting.

41. The method of claim 37, further comprising contacting said seed with a DNA protectant.

42. The method of claim 41, wherein said DNA protectant is selected from the group of an antioxidant and a histone.

43. A sample of representative seeds of a Durum wheat plant having a partially or fully multiplied genome being at least as fertile as a tetraploid Durum wheat (Triticum durum) plant isogenic to said genomically multiplied Durum wheat plant when grown under the same conditions and being of the same developmental stage, wherein said sample has been deposited under the Budapest Treaty at the NCIMB under NCIMB 42002.

44. A sample of representative seeds of a Durum wheat plant having a partially or fully multiplied genome being at least as fertile as a tetraploid Durum wheat (Triticum durum) plant isogenic to said genomically multiplied Durum wheat plant when grown under the same conditions and being of the same developmental stage, wherein said sample of said Durum wheat plant having said partially or fully multiplied genome has been deposited under the Budapest Treaty at the NCIMB under NCIMB 42002.

45. A durum wheat seed obtainable according to the method of claim 37.
